(19) United States
(12) Patent Application Publication
(10) Pub. No.: US 2025/0197316 A1
(43) Pub. Date: Jun. 19, 2025

(54) METAL ORGANIC FRAMEWORK-IMMOBILIZED CR-MONOPHOSPHINE COMPLEX CATALYZED ETHYLENE PENTAMERIZATION TO SELECTIVELY PRODUCE 1-DECENE

(71) Applicant: Hamad Bin Khalifa University (HBKU), Doha (QA)

(72) Inventors: Sherzod T. Madrahimov, Doha (QA); Veeranna Yempally, Doha (QA)

(21) Appl. No.: 18/975,682

(22) Filed: Dec. 10, 2024

Related U.S. Application Data
(60) Provisional application No. 63/610,250, filed on Dec. 14, 2023.

(51) Int. Cl. C07C 2/36; B01J 31/16; B01J 31/24; C07F 9/50

(52) U.S. Cl. CPC C07C 2/36; B01J 31/1691; B01J 31/24; C07F 9/5045; B01J 2231/20; B01J 2531/004; B01J 2531/62; C07C 2531/24

(57) ABSTRACT

The Disclosed Technology relates to a Metal Organic Framework (MOF)-immobilized monophosphine-chromium catalyst that is able to selectively catalyze the preparation of highly valuable 1-decene from ethylene. The catalyst has high activity and is almost exclusively selective for production of 1-decene. The Disclosed Technology immobilizes a monophosphine chromium catalyst on a catalyst support consisting of a Metal Organic Framework (MOF). This enables formation of an underligated Cr-monophosphine complex on the surface of the MOF, which is stabilized through immobilization. The underligated nature of the MOF enables the unique selectivity observed for the system. Notably, the Disclosed Technology can be scaled up to selectively produce 1-decene in industrial settings. Additionally, the Disclosed Technology can be optimized further and also modified (in terms of ligand and active metal) to achieve high selectivity for other ethylene oligomers.

### PRIORITY CLAIM AND CROSS-REFERENCE TO RELATED APPLICATIONS

The present application claims the benefit of U.S. Provisional Application No. 63/610,250 filed Dec. 14, 2023, which is incorporated herein by reference in its entirety.

### BACKGROUND

The current solutions for production of 1-decene involve the use of ethylene oligomerization catalysts that are not selective for a particular ethylene oligomer, but produce a Schulz-Flory distribution of ethylene oligomers, that depends on the reaction conditions.

### SUMMARY

According to one non-limiting aspect of the present disclosure, an exemplary embodiment of a Metal Organic Framework (MOF)-immobilized Cr-monophosphine complex catalyzed ethylene pentamerization to selectively produce 1-decene.

According to a second non-limiting aspect of the present disclosure, an exemplary embodiment of a method of producing a Metal Organic Framework (MOF)-immobilized Cr-monophosphine complex catalyzed ethylene pentamerization to selectively produce 1-decene.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. In addition, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 shows a chemical reaction, according to an example embodiment of the present disclosure.

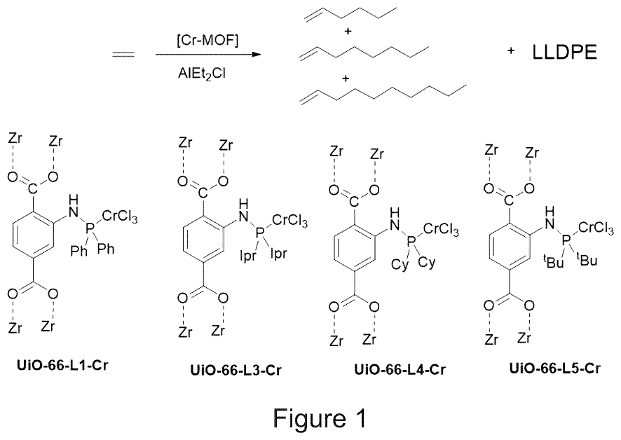

### DETAILED DESCRIPTION

The present disclosure generally relates to the production of highly valuable 1-decene from ethylene with exclusive selectivity and high turnover numbers. Current industrial catalysts do not produce 1-decene selectively and the product needs to be separated from the mixture of ethylene oligomers.

The Disclosed Technology relates to an MOF-immobilized monophosphine-chromium catalyst that is able to selectively catalyze the preparation of highly valuable 1-decene from ethylene. The catalyst has high activity and is almost exclusively selective for production of 1-decene. The Disclosed Technology immobilizes a monophosphine chromium catalyst on a catalyst support consisting of a Metal Organic Framework (MOF). This enables formation of an underligated Cr-monophosphine complex on the surface of the MOF, which is stabilized through immobilization. The underligated nature of the MOF enables the unique selectivity observed for the system. Notably, the Disclosed Technology can be scaled up to selectively produce 1-decene in industrial settings. Additionally, the Disclosed Technology can be optimized further and also modified (in terms of ligand and active metal) to achieve high selectivity for other ethylene oligomers.

### Synthesis of Mono-(Diphenylphosphino) Amino Terepthalate Ligand (Me-L1)

The ligand was synthesized according to the literature procedure with slight modification. In a 100 mL two neck flask, Dimethyl amino terephthalate (1.04 g, 4.00 mmol), and triethyl amine (2.00 g, 20.0 mmol) were dissolved in 20 mL of dichloromethane under N₂ atmosphere. Then the reaction was cooled to zero° C. using an Ice bath. To the resulting suspension, Chlorodiphenylphosphine (2.20 g, 10.0 mmol) was added dropwise over a period of 15 min. The reaction mixture stirred at room temperature for 18 h. The solution was dried through rotary evaporation and the residue obtained was treated with 20 mL cold ethanol, and then white precipitate obtained was filtered. The white precipitate was washed with cold ethanol (3.00 mL*3 times) to obtain pure white solid (1.29 g, yield: 75.0%). The product was further purified by flash column chromatography employing eluting mixture of hexane and ethylacetate (9:1 ratio, with few drops of triehylamine). H-NMR (400 MHZ, CDCl3) δ 8.72 (d, 1H), 8.27 (d, 1H), 7.99 (d, 1H), 7.50-7.36 (m, 10H), 3.88 (d, 6H). 31P-NMR (400 MHZ, CDCl3-d6) δ 24.7 ppm. MW: 393.38 2.03.

### Synthesis of Mono-(Diphenylphosphino) Amino Terepthalic Acid (L1)

The mono-(diphenylphosphino) amino terephthalate (0.80 g, 2.03 mmol) was suspended in a 30 mL solution of 1:1:1 mixture of (THF:MeOH:H2O) in a 100 mL round bottom flask. 0.70 g of KOH (12.0 mmol) pellets were added to the above suspension and stirred at room temperature for 12 h, and then slowly warmed to 40° C., and stirred at 40° C. for additional 2 h. The clear yellow solution obtained was dried through rotary evaporation, and resulting solution was cooled to zero° C. using an Ice bath, and 2.0 M aq HCL solution was added dropwise until yellow color precipitate was observed. The yellow solid was filtered through glass frit, and washed with deionized water and finally with cold methanol to obtain light yellow solid (0.75 g, 99% yield). H-NMR (400 MHZ, DMSO-d6) δ 8.64 (m, br, 2H), 7.76 (d, 2H), 7.38-7.26 (m, 10H). 31P-NMR (400 MHZ, DMSO-d6) δ 18.7 ppm. MW: 365.08.

### Synthesis of UiO-66-NHPPh2

To a solution of UiO-66 (50.0 mg) in DMF (10.0 mL), 50.0 mg of NHPPh2 ligand was added to form a solution mixture and was then sonicated for 15 mins. Then the solution was heated at 80° C. overnight until the powder changes color from white to pale yellow. After ligand exchange, the powder was washed with DMF (10 mL×3) and then soak in 0.1M HCl DMF solution at 80° C. for 8 hours to remove the excess surface coordinated ligands. Finally, the powder was washed with DMF (10 mL×3), acetone (10 mL×3) and then dried under vacuum at 100° C. overnight. Ligand exchange ratio was confirmed by digest NMR. Similar procedure was followed for the synthesis of MOF with the Ligands L2-L5.

Details of digested NMR with 31P external standard:

Metal-organic framework (MOF) materials were digested for 1H NMR and 31P NMR analysis by sonicating a small amount of MOF material (˜3 mg) in conc. D₂SO₄ (5 drops) until all of the MOF disintegrated and a brown heterogeneous solution formed. Upon addition of (CD₃)₂SO (0.5 mL) to this solution and further sonication, a homogeneous pale yellow solution forms, which was Cr (III) metal organic frameworks appended with monophoshine-amino ligands have been employed in selective catalytic conversion of ethylene into 1-decene and linear alpha olefins. The selectivity to linear alpha olefins is dependent on two different factors. 1) The solvent used during the reaction, 2) the steric and electronic nature of the monophosphine ligand appended on the MOF.

The Bulky t-Bu and cyclohexyl groups on the NH-linker of MOF prohibited formation of lower chain linear alpha olefins, mostly selective to formation of linear lower molecular weight polyethylene. Whereas the less bulky Phenyl and Isopropyl groups on the NH-Linker of MOF favored formation of 1-decene selectively with higher turnover numbers and with negligible formation of polyethylene products. Interestingly, stronger coordinating solvent favored the formation of small chain linear alpha olefins, whereas non-coordinating solvents like Toluene, Heptane resulted in higher yields of LLDPE. This selectivity dependence on Solvent, nature of metal and nature of ligand framework has interesting application in ethylene oligomerization for selective transformation of ethylene into desired liquid products.

At the conclusion of the reaction the production of 1-decene was observed via GC-FID spectroscopy and the retention time of the FID peak was matched with a commercial sample of 1-decene. The activity and selectivity of the reaction was calculated by comparison of the 1-decene FID peak area to the FID peak area of an internal standard added to the reaction mixture at the beginning of the reaction. The outcomes of the GC-FID analysis are tabulated below.

| | Time | Solvent | 1-c6 | 1-c8 | 1-decene | C12-C16 | Polymer (mg) | | Total TOF(h − 1) |
|---|---|---|---|---|---|---|---|---|---|
| UiO-66-L1 | 1 h | THF | 8.0 | 15.0 | 77.0 | 0.0 | 3.0 | | 30300 |
| UiO-66-L1 | 1 h | Toluene | 0.0 | 0.0 | 0.0 | 99 | 15 | mg | 61500 |
| UiO-66-L1 | 1 h | MCH | — | — | — | — | 55 | mg | |
| UiO-66-L1 | 1 h | Heptane | — | — | — | — | 175 | mg | |
| UiO-66-L3 | 1 h | Toluene | 0.0 | 10.0 | 87.0 | 2.0 | 15.0 | | 130250 |
| UiO-66-L3 | 1 h | THF | 0.0 | 0.0 | 99.0 | 0.0 | 10.0 | | 27500 |
| UiO-66-L5 | 1 h | THF | 54.0 | 46.0 | 0.0 | 0.0 | 5.0 | mg | 3830 |
| UiO-66-L5 | 1 h | Toluene | — | — | — | — | 120 | mg | |
| UiO-66-L4 | 1 h | THF | — | — | — | — | 100 | mg | |
| UiO-66-L4 | 1 h | Toluene | — | — | — | — | 85 | mg | |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims

The invention is claimed as follows:

1. A method of producing a Metal Organic Framework (MOF)-immobilized Cr-monophosphine complex catalyzed ethylene pentamerization to selectively produce 1-decene, the method comprising:
synthesizing mono-(diphenylphosphino) amino terepthalate ligand (Me-L1),
sonicating an amount of metal-organic framework (MOF) materials and mono-(diphenylphosphino) amino terepthalate ligand (Me-L1) in an amount of concentrated D₂SO₄ until all of the amount of metal-organic framework (MOF) materials disintegrate and a brown heterogenous solution forms,
adding (CD₃)₂SO to the brown heterogenous solution to result in a resulting solution,
sonicating the resulting solution until a homogenous pale yellow solution forms,
using the pale yellow solution in selective catalytic conversion of ethylene into 1-decene.

2. The method of claim 1, wherein the amount of metal-organic framework (MOF) materials is 3 mg.

3. The method of claim 1, wherein the amount of concentrated D₂SO₄ is 5 drops.

4. The method of claim 1, wherein the amount of (CD₃)₂SO is 5 mL.

5. The method of claim 1, wherein synthesizing mono-(diphenylphosphino) amino terepthalate ligand (Me-L1) comprises:
dissolving 1.04 g. of 4.00 mmol Dimethyl amino terephthalate and 2.00 g. of 20.0 mmol triethyl amine in 20 mL of dichloromethane under N₂ atmosphere in a 100 mL two neck flask to result in a reaction,
cooling the reaction to 0° C. using an ice bath to result in a suspension,
adding 2.20 g. of 10.0 mmol Chlorodiphenylphosphine to the suspension dropwise over a period of 15 min resulting in a reaction mixture,
stirring the reaction mixture at room temperature for 18 hour resulting in a solution,
drying the solution through rotary evaporation resulting in a residue,
treating the residue with 20 mL of cold ethanol resulting in a white precipitate,
filtering the white precipitate,
washing the white precipitate three times with 3.00 mL of cold ethanol resulting in a pure white solid, wherein the pure white solid is 1.29 g. of mono-(diphenylphosphino) amino terepthalate ligand (Me-L1) at a 75% yield, and
further purifying the pure white solid by flash column chromatography employing eluting mixture of hexane and ethylacetate at a 9:1 ratio and with a few drop of triehylamine.

6. A method of producing a Metal Organic Framework (MOF)-immobilized Cr-monophosphine complex catalyzed ethylene pentamerization to selectively produce 1-decene, the method comprising:
synthesizing mono-(diphenylphosphino) amino terepthalic acid (L1),
sonicating an amount of metal-organic framework (MOF) materials and mono-(diphenylphosphino) amino terepthalic acid (L1) in an amount of concentrated D₂SO₄ until all of the amount of metal-organic framework (MOF) materials disintegrate and a brown heterogenous solution forms,
adding an amount of (CD₃)₂SO to the brown heterogenous solution to result in a resulting solution,
sonicating the resulting solution until a homogenous pale yellow solution forms,
using the pale yellow solution in selective catalytic conversion of ethylene into 1-decene.

7. The method of claim 6, wherein the amount of metal-organic framework (MOF) materials is 3 mg.

8. The method of claim 6, wherein the amount of concentrated D₂SO₄ is 5 drops.

9. The method of claim 6, wherein the amount of (CD₃)₂SO is 5 mL.

10. The method of claim 6, wherein synthesizing mono-(diphenylphosphino) amino terepthalic acid (L1) comprises:
suspending 0.80 g of 2.03 mmol mono-(diphenylphosphino) amino terephthalate in a 30 mL solution of 1:1:1 mixture of THF:MeOH:H2O in a 100 mL round bottom flask to result in a suspension,
adding 0.70 g of 12.0 mmol KOH pellets to the suspension and stirring at room temperature for 12 hours,
slowly warming the suspension to 40° C. and stirring at 40° C. for 2 hours to result in a clear yellow solution,
drying the clear yellow solution through rotary evaporation to result in a solution,
cooling the solution to 0° C. using an ice bath,
adding 2.0 M aqueous HCL solution dropwise until a yellow precipitate is observed,
filtering the yellow precipitate through a glass grit,
washing the yellow precipitate with deionized water, and
washing the yellow precipitate with cold methanol to obtain 0.75 g. of a light yellow precipitate at 99% yield.

11. A method of producing a Metal Organic Framework (MOF)-immobilized Cr-monophosphine complex catalyzed ethylene pentamerization to selectively produce 1-decene, the method comprising:
synthesizing UiO-66-NHPPh2,
sonicating an amount of metal-organic framework (MOF) materials and UiO-66-NHPPh2 in an amount of concentrated D₂SO₄ until all of the amount of metal-organic framework (MOF) materials disintegrate and a brown heterogenous solution forms,
adding an amount of (CD₃)₂SO to the brown heterogenous solution to result in a resulting solution,
sonicating the resulting solution until a homogenous pale yellow solution forms,
using the pale yellow solution in selective catalytic conversion of ethylene into 1-decene.

12. The method of claim 11, wherein the amount of metal-organic framework (MOF) materials is 3 mg.

13. The method of claim 11, wherein the amount of concentrated D₂SO₄ is 5 drops.

14. The method of claim 11, wherein the amount of (CD₃)₂SO is 5 mL.

15. The method of claim 11, wherein synthesizing UiO-66-NHPPh2 comprises:
adding 50.0 mg of NHPPh2 ligand a solution of 50.0 mg of UiO-66 in 10.0 mL of DMF to result in a solution mixture,
sonicating the solution mixture for 15 mins,
heading the solution mixture at 80° C. overnight, wherein the solution mixture is heated until a powder in the solution mixture changes from white to pale yellow,
washing the powder with 10 mL of DMF three times,
soaking the powder in a 0.1M HCl DMF solution at 80° C. for 8 hours,
washing the powder with 10 mL of DMF three times and with 10 mL of acetone three times, and
drying the powder under a vacuum at 100° C. overnight.
